(19) United States
(12) Patent Application Publication
(10) Pub. No.: US 2022/0175531 A1
(43) Pub. Date: Jun. 9, 2022

(54) Grayson Bionic Penis (GBP)

(71) Applicant: Michael C. Grayson, Hollis, NY (US)

(72) Inventor: Michael C. Grayson, Hollis, NY (US)

(21) Appl. No.: 17/116,043

(22) Filed: Dec. 9, 2020

(51) Int. Cl. A61F 2/26

(52) U.S. Cl. CPC A61F 2/26; A61F 2250/0078

(57) ABSTRACT

This prosthesis is likely to be implanted surgically in males for the treatment of erectile disfunction, higher degree of female satisfaction, erection improvement, greater erection management, cosmetic enhancement, and or premature ejaculation concerns and it has at least one flexible semi-rigid inflatable computerized remote controlled software driven sleeve bladder that can be inflated to create an erection or deflated on demand, implanted inside the inner wall and encompassing the circumference of the penis, and can incorporate at least one rechargeable vibrator device, at least one flexible inflatable computerized remote controlled cylindrical bladder, implanted inside the penis body, that can be inflated to create an erection, a computerized remote controlled fluid-filled reservoir implanted under the abdominal wall, a wirelessly rechargeable computerized remote controlled pump and release valve placed under the abdominal wall with a subcutaneous wireless recharging system. Each device is controlled by an erection management system (EMS) and corresponding software.

### BACKGROUND

Prosthetic for Penis

There are two main types of penile implants, semirigid and inflatable. This device incorporates both. Each type of penile implant works differently and has various pros and cons, by combining the two the user get the benefits of both worlds while negating the negative attributes. The present invention relates to penile prostheses for treating impotence to erection, premature ejaculation, cosmetic or general dissatisfaction with erection management. This is the world's first Erection Management System (EMS). It relates more particularly to a computer controlled wirelessly rechargeable remotely controlled inflatable penis implants with built in vibrator. Used in some cases of erectile dysfunction for which the patient does not respond to more conventional therapy, for patients who suffer from premature ejaculation and cannot sustain prolong erections, for patients who are generally unhappy with the length of time that they can maintain erection and prefer more control over their sexual experience, and for those patients who wish to increase the overall cosmetic appeal of their penis, this surgical implantation of a computer controlled penile prosthesis may be the only practical way to remedy their feelings of powerlessness, while providing a solution that does not alert the sexual partner that the user has a prosthetic. The Erection Management System software provides the user with several options to control the sexual experience including but not limited to: size, stiffness, length, circumference, timer, and vibration.

Several types of penile prostheses have been used previously. The first type consists of a cylindrical bladder which is made of one or two rods of suitable stiffness, each of which is implanted surgically into a cavernous body of the penis. A disadvantage of this rod implant is that the rigidity of the rods makes it difficult to implant rods of sufficient diameter for the penis to have a normal circumference in the erect state. Another type of penile prosthesis that is available on the market is the inflatable prosthesis which in the most common form has two relatively long and expandable tubes that are surgically implanted in the cavernous body of the penis. Each of the two tubes is connected by tubing to an inflating fluid pressure bulb which is implanted at any point in the body. The expandable tubes can collapse, so that they can be implanted easily and can be inflated to increase the circumference of the penis so that a normal erection is achieved. In this device the two rods will be semirigid and inflatable to alleviate this problem. In addition, because of the large volume of liquid that is required to pressurize and make rigid inflatable tubes, the reservoir must have a relatively large size. This prosthesis has a relatively small dimension, and the pressurizing fluid is supplied from a single reservoir which is implanted in the abdominal cavity.

This device also incorporates Another type of inflatable prosthesis for a penis that can increase the size, circumference and stiffness. It comprises an implant known as a sleeve bladder. Most traditional sleeves again are rigid and cannot be customized. Each sleeve bladder implant in this device is made of an expandable inflatable semi rigid material for supporting the implant which incorporates a balloon-shaped expandable and collapsible portion which encompasses the penis and which is inflated so as to simulate an erection. Together with the cylindrical bladder the user can achieve unparalleled size and stiffness.

Patents have recently been published describing penis implants that are inflatable. Such implants are cylindrical members having integrated pumps, reservoirs and pressure chambers. The pressure chambers are not so great that it is sufficient to transfer only a small amount of fluid to make the pressure chamber rigid. However, these inflatable implants do not increase the circumference or size of the penis and delivery only a relatively small amount of rigidity. A large number of patients suffering from erectile dysfunction are not satisfied with prior implants or inflatable penis implants because either they do not increase the circumference or size of the penis or they are not rigid enough to satisfy the user. It would obviously be desirable to have a simple implant by which the penile circumference and size can be increased and preferably an implant which is has two ways to increase rigidity and stiffness of the penis.

### SUMMARY

It is an object of the present invention to provide an improved penile inflatable prosthesis having means for giving the patient the ability to increase the penile circumference, size, hardness and the length. One of the aims of the invention is also to propose an implant that adjusts the circumference, hardness and length of the penis in an adjustable manner without the use of a large pressure bulbs. The common pressure bulb which can be implanted in the scrotal bag must be manually adjusted and pumped by the user. Such a process is cumbersome and detracts from the overall sexual experience. For this purpose, the present invention comprises a remote controlled, software driven rechargeable electric pump, that can be inflated discretely, quickly and without disrupting the sexual process. The implant according to the present invention differs from other devices because it offers a subcutaneous recharging system to power the prosthetics.

The implant also includes a vibrator to increase the pleasure and effectiveness of the user. This at least one vibrator device can be place in several locations including but not limited to along the sleeve bladder.

In a preferred embodiment, the cylindrical bladder member is provided with a relatively rigid proximal end which is implantable at the root end of the cavernous body to anchor and support the implant and the implant. The distal portion cylindrical member contains the inner and outer chambers and is capable of being implanted in a corpus cavernosum the pendulous portion of the penis. The most flexible distal portion causes minimal irritation to the penile tissues and allows the pendulum portion of the penis to assume a normal position when the implant is not inflated.

In a preferred embodiment, the sleeve bladder is shaped to fit around the penis. The semi rigid bladder portion causes minimal irritation to the penile tissues and allows the pendulum portion of the penis to assume a normal position when the implant is not inflated.

The penis implant according to the present invention has a unique feature over previously available inflatable implants in that it allows for adjustable adjustment of the penile circumference, size and stiffness. This is an important benefit for patients who wish to have a normally erect penis. The implant according to the present invention can be implanted and after the patient has completely healed, the device can be pressurized with liquid from the reservoir to simulate an erection of the penis. However, if the penile circumference, size or stiffness is less than desired, its circumference, size and stiffness can be effectively increased to a more acceptable level by transferring liquid from the reservoir to either of the dual bladder system. This is achieved by programing the electronic device which controls the rechargeable pump and valve. Other goals, advantages and features will appear on reading the description of several modes of embodiment of the invention, made in a non-limiting manner and with reference to the accompanying drawing

### BRIEF DESCRIPTION OF THE DRAWINGS

FIG. 1 is a partial sectional view of the penis prosthesis according to the present invention, in the state without pressure, as it is implanted surgically in a male subject;

FIG. 2 is a cross-sectional view of the penis prosthesis along with the REMOTE CONTROLLED COMPUTERIZED ERECTION MANAGEMENT SYSTEM AND CORRESPONDING SOFTWARE device;

FIG. 3 is a sectional view of the WIRELESS RECHARGING SYSTEM;

FIG. 4 is a view of the ELECTRONIC DEVICE that controls the prosthesis;

FIG. 5 is a view of the INFLATABLE SLEEVE BLADDERS;

FIG. 6 is a view of the SLEEVE bladder PLACEMENT.

### DETAILED DESCRIPTION

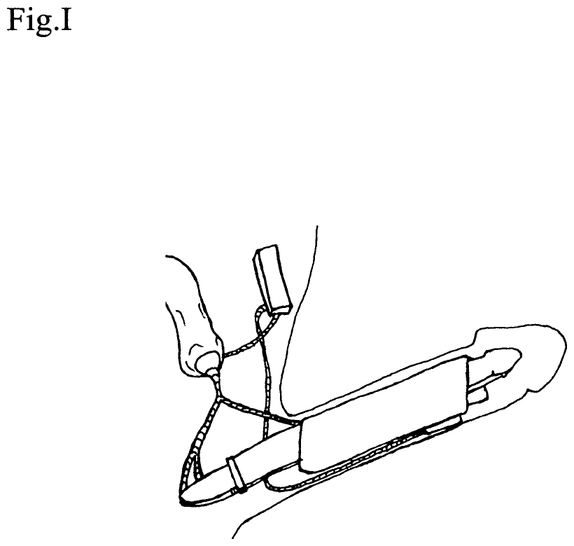

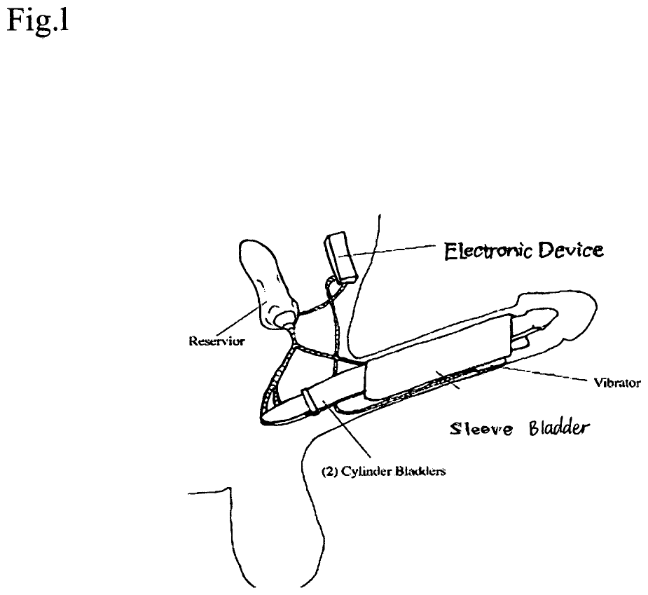

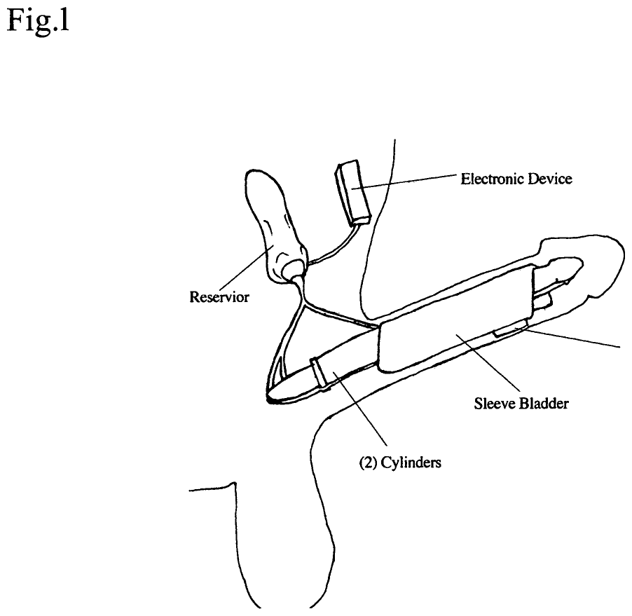

Referring now to FIG. 1, it will be seen that when the bladder chambers are pressurized, the flexible and soft distal portion of the member is supported by the pressure bladder chambers and the penis has a substantially normal erection, this normal erection can be greatly modified if desired to give above average penis circumference, size and stiffness. In the erection form, the pressure bladder chamber is filled and can be completely filled to the maximum position, the reservoir contains pressurizing fluid. The pressure bladder chambers are made rigid simply by using the remote control and activating the electronic pump, which opens the valve and allows the pressurizing fluid to fill the bladders. The remote control can also activate the vibrator.

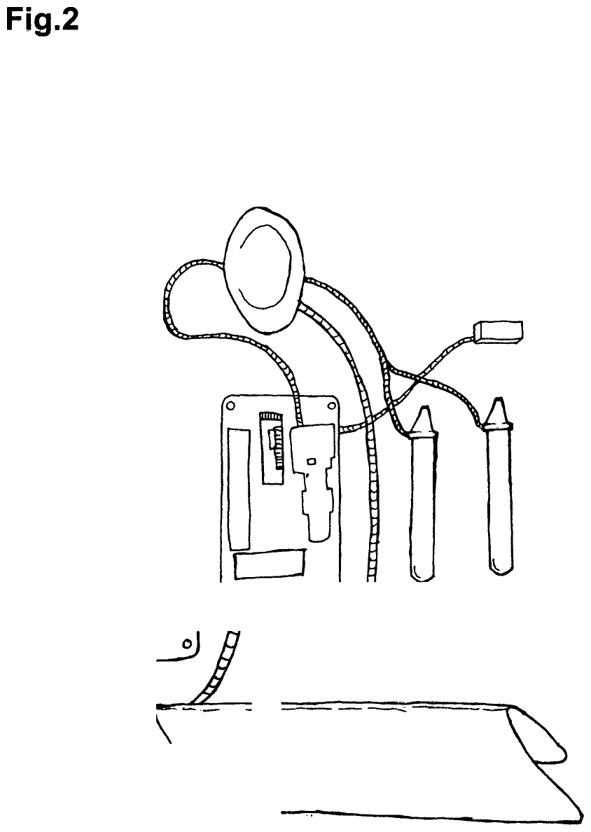

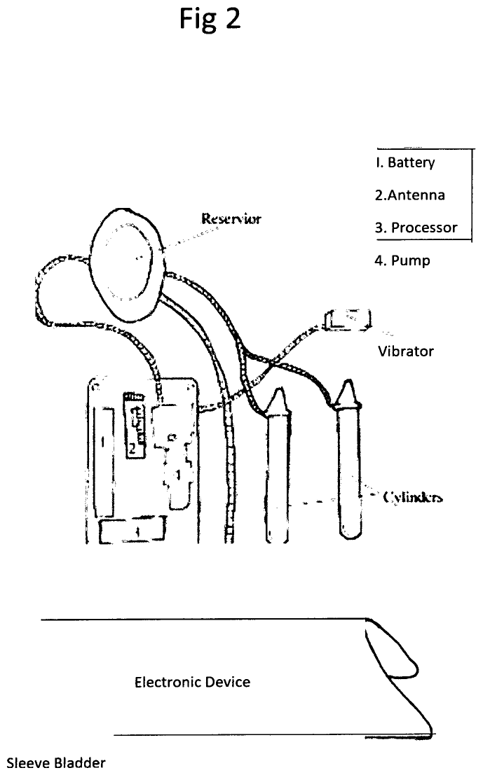

Referring now to FIG. 2, As seen in the drawing, the prosthesis is controlled by a remote-control device. This device is controlled by an Erection Management System software. The remote activates an electronic device which houses the electric pump and valve. The remote allows the user to control: size, stiffness, circumference, the vibrator each of at least two pressure bladder, it also has a timer control and battery indicator. The rods of the two cylinder bladder implants can diverge laterally to match to the anatomy and ensure the lateral stability of the penis. In the preferred embodiment, all parts and components of the prosthesis are made of medical silicone rubber which is non-reactive, non-toxic and well tolerated by adjacent body tissues. Silicone rubber is preferred because it is very resistant to wear and tear and remains functional for long periods of time. However, other suitable materials may be used, if desired.

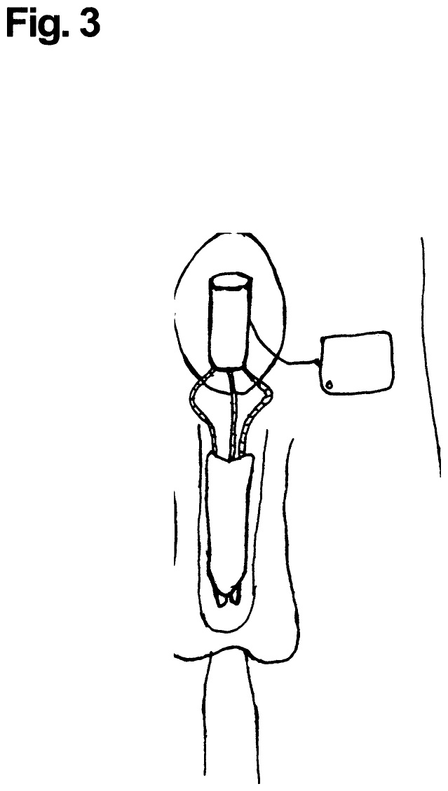

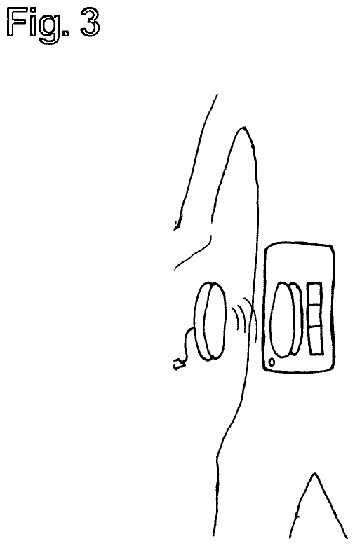

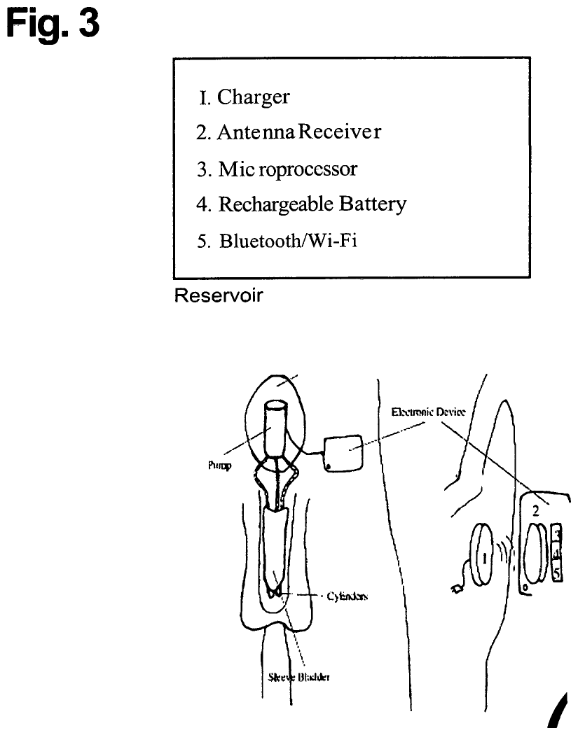

Referring more particularly now to FIG. 3, which shows the subcutaneous recharging system. This wireless recharging system charges the battery on the electronic device. The electronic device in turn powers the pump, valve and vibrator.

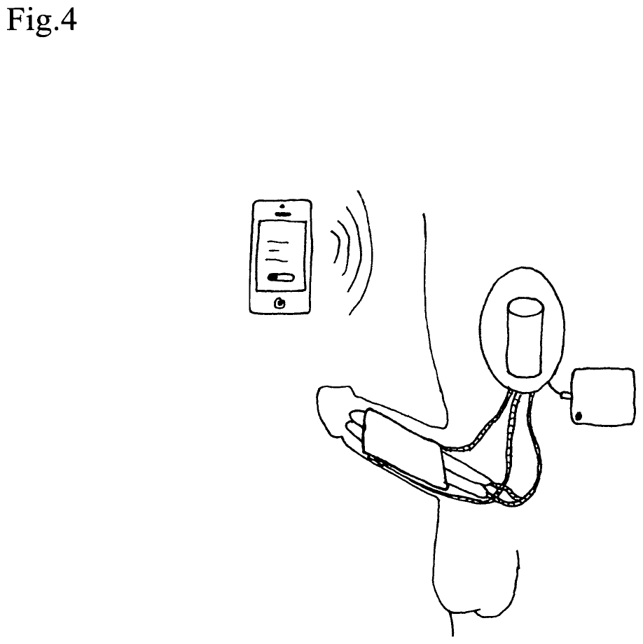

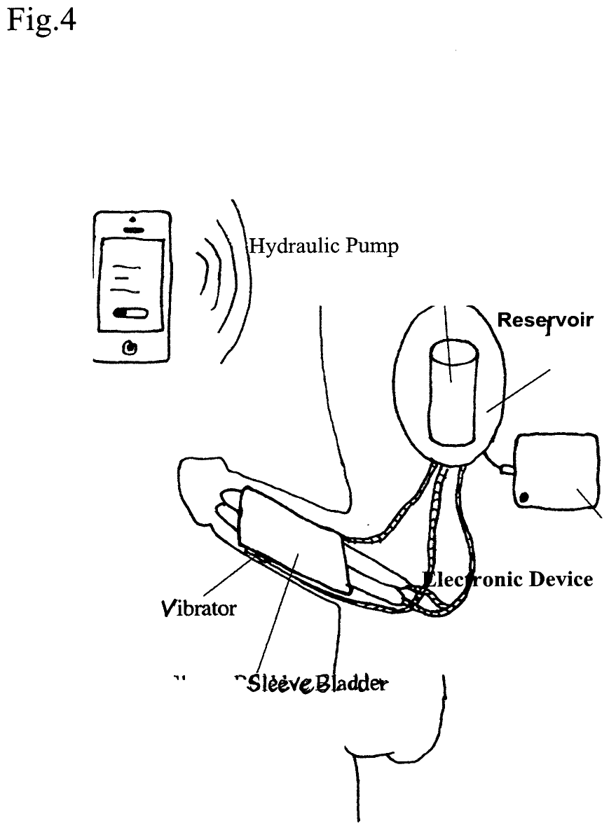

Referring more particularly now to FIG. 4, which shows the electronic device in detail and the devices that it controls.

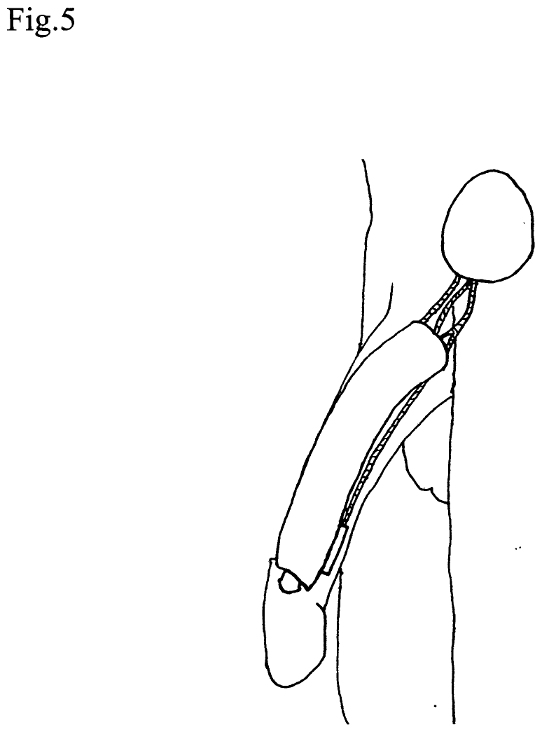

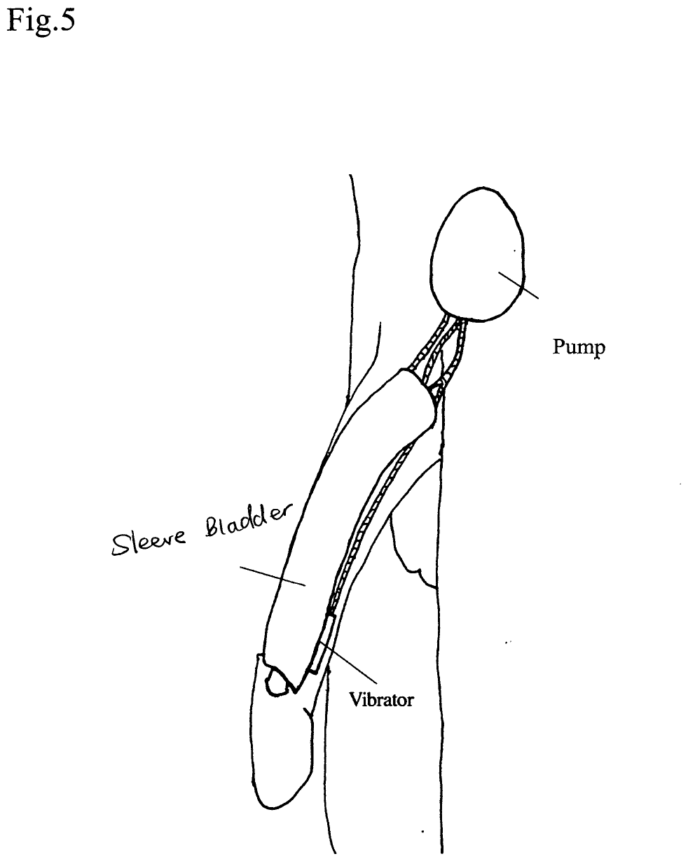

Referring more particularly now to FIG. 5, which shows the inflatable sleeve bladder. This device encompasses the penis and adds a degree of stiffness to the penis which can supplement the bladder inserts inside the penis body. The sleeve is preferably made of silicone elastomer covered with woven or knitted fabric which allows a limited predetermined expansion so as to allow the penis to lie down and withstand the pressure in such a way that the tunica albuginea or albuginea is not distended. Alternatively, the sleeve may also be made of a material which does not distend axially or longitudinally. The sleeve has a built-in bladder that is inflatable. The diameter of the sleeve is sufficient to form a functional pressure chamber. The use of a sleeve material which does not distend or distend only to a limited value makes it possible to increase the fluid pressure in the chamber to the desired high level with only a minimum of pressurizing fluid. The sleeve may be made of an unreinforced silicone rubber or any functionally equivalent or superior material.

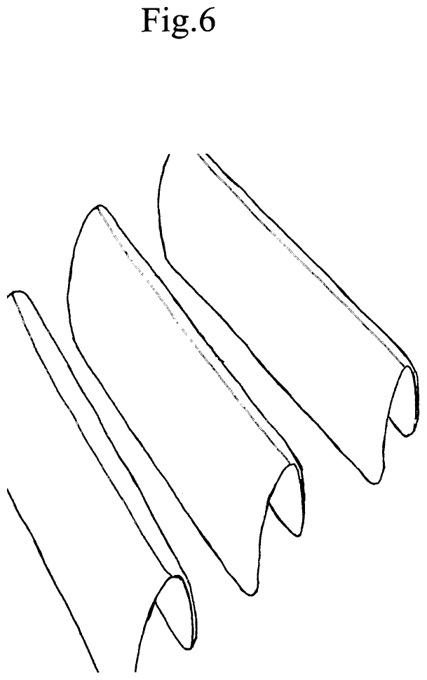

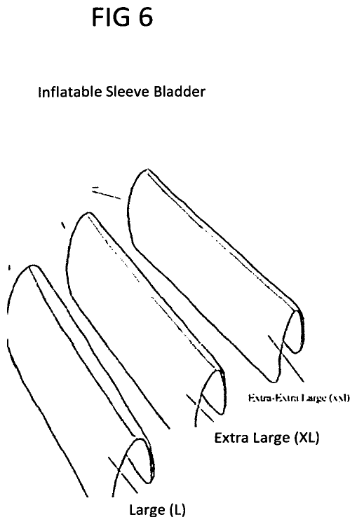

Referring more particularly now to FIG. 6, it is seen that when the bladder chamber of the implant is no longer pressurized, the relatively flexible and soft distal portion of the member allows the penis to take a state substantially normal and flaccid. In the preferred embodiment, as shown in FIG. 6, the sleeve bladder is housed beneath the penis skin near the surface. The use of the implant for increasing the circumference of the penis will be described. As shown in FIG. 6, once the first valve is opened by the remote control, pressurizing liquid will flow via the passage into the sleeve bladder and through the passage forcing it to expand outward and to increase the circumference, stiffness and size of the penis. When the valve activation ceases, the passage is closed and the liquid which has been added is retained in the chamber. The additional liquid of the reservoir can be used to fill the other chambers. The computerized pump which is better represented in FIG. 6, in such a way that additional fluid can be added or subtracted on demand. This may be necessary if the transfer of fluid to increase the circumference to the required level is too great to allow the desired erection. The valve as shown in FIG. 4 is used to automatic the sealing, filling and releasing process. When it is desired to return the implant to the non-pressurized form shown in FIG. 6, the remote valve is opened in order to allow the pressurizing fluid to be returned to the reservoir chamber via the passage. When an adequate amount of fluid has been returned to storage in the reservoir, the penis returns to the flaccid state.

Although there is shown in the drawing a single penis implant, the complete penile prosthesis normally includes at least two separate penis implants, each connected to a common pressurizing pump and valve which can be surgically implanted in the abdominal region.

Of course, the present invention is not limited to the embodiments described and shown and is capable of numerous variants accessible to those skilled in the art without departing from the spirit of the invention.

1. An electronic computer controlled software driven remotely operated wirelessly rechargeable penile prosthesis characterized in that it comprises: (a) at least one elongated flexible semi rigid inflatable customizable cylindrical bladder member capable of being implanted in the cavernous body of the pendular part of the penis; (b) at least one flexible semi rigid inflatable customizable sleeve bladder that surrounds the penis and is located under the skin; (c) an optional vibrator device positioned inside the penis area; (d) a software controlled rechargeable remote electric device which houses at least one pump and valve disposed outside the member for supplying fluid under pressure to said pressure bladder chambers and recharges the optional vibrator device; (c) means connecting the pressure chamber and the pump so that pressurizing fluid can be introduced into and removed from the pressure chambers; and valve means in the member for controlling flow through the passage between the bladder chambers, so that the amount of fluid in the chambers and the circumference, size and stiffness of a penis in which the implant is implanted can be increased and customized in transferring fluid from the pump (e) at least one Erection Management System; (f) at least one computer controlled rechargeable subcutaneous recharging system.

2. The device according to claim 1, wherein at least one of said sleeve bladders is made of a semi rigid expandable and collapsible portion which encompasses the penis, and which is inflated so as to simulate an erection.

3. The device according to claim 1, wherein said device adjusts the circumference, hardness and length of the penis in an adjustable manner automatically without the use of a large manually operated pressure bulb.

4. The device according to claim 1, wherein the rechargeable pump can be inflated and deflated discretely and quickly without disrupting the sexual process or advertising the fact that the user has an implant.

5. The device according to claim 1, wherein at least one of said vibrator devices increases the pleasure and effectiveness of a user.

6. The device according to claim 1, wherein at least one of said vibrator devices can be placed in several locations including but not limited to along at least one of said sleeve bladders.

7. The device according to claim 1, wherein a most flexible distal portion causes minimal irritation to the penile tissues and allows the pendular part of the penis to assume a normal position when said organ is not inflated.

8. The device according to claim 1, wherein the semi rigid bladder portions cause minimal irritation to the penile tissues and allows the pendular part of the penis to assume a normal position when said organ is not inflated.

9. A subcutaneous method for controlling wirelessly rechargeable penile prosthesis characterized in that it comprises: (a) at least one elongated flexible semi rigid cylindrical member capable of being implanted in the cavernous body of the pendular part of the penis, said organ comprising a customizable inflatable sleeve bladder; (b) a software controlled remote pump and valve disposed outside the member for supplying fluid under pressure to said pressure bladder/chamber; (c) means connecting the pressure chamber and the pump so that pressurizing fluid can be introduced into and removed from the pressure chamber; and valve means in the member for controlling flow through the passage between the bladder chambers, so that the amount of fluid in the chambers and the circumference of a penis in which the implant is implanted can be increased in transferring fluid from the pump (d) at least one inflatable sleeve bladder that surrounds the penis; at least one vibrator device; (e) at least one Erection Management System software device; (f) at least one computer controlled rechargeable pump and valve; at least one subcutaneous recharging system.

10. The method according to claim 9, wherein at least one of said sleeve bladders is made of an expandable and collapsible portion which encompasses the penis and which is inflated so as to simulate an erection.

11. The method according to claim 9, wherein said device adjusts the circumference, hardness and length of the penis in an adjustable manner automatically without the use of a large pressure bulb.

12. The method according to claim 9, wherein the rechargeable pump can be used to inflate and deflate the penis discretely and quickly without disrupting the sexual process or alerting the sexual partner that the user has a prosthetic.

13. The method according to claim 9, wherein at least one of said vibrator devices increases the pleasure and effectiveness of a user.

14. The method according to claim 9, wherein at least one of said vibrator devices can be placed in several locations including but not limited to along at least one of said sleeve bladders.

15. The method according to claim 9, wherein a most flexible distal portion causes minimal irritation to the penile tissues and allows the pendular part of the penis to assume a normal position when said organ is not inflated.

16. The method according to claim 9, wherein the semi rigid bladder portion causes minimal irritation to the penile tissues and allows the pendular part of the penis to assume a normal position when said organ is not inflated.

17. A non-transitory computer-readable storage medium having stored thereon instructions that, when being executed by a processor, cause the processor to control:
(a) at least one elongated flexible semi rigid cylindrical member capable of being implanted in the cavernous body of the pendular part of the penis, said organ comprising a customizable inflatable sleeve bladder; (b) a software controlled remote pump and valve disposed outside the member for supplying fluid under pressure to said pressure bladder/chamber; (c) means connecting the pressure chamber and the pump so that pressurizing fluid can be introduced into and removed from the pressure chamber; and valve means in the member for controlling flow through the passage between the bladder chambers, so that the amount of fluid in the chambers and the circumference of a penis in which the implant is implanted can be increased in transferring fluid from the pump (d) at least one inflatable sleeve bladder that surrounds the penis; at least one vibrator device; (e) at least one Erection Management System; (f) at least one computer controlled rechargeable pump and valve; at least one subcutaneous recharging system.
